(19) United States
(12) Patent Application Publication
(10) Pub. No.: US 2026/0054009 A1
(43) Pub. Date: Feb. 26, 2026

(54) NEEDLE FREE CONNECTOR CONTROL VALVE

(71) Applicant: CareFusion 303, Inc., San Diego, CA (US)

(72) Inventors: Jason Andrew WINE, Brea, CA (US); Aman DESAI, Bengaluru (IN); Kowshika K, Tirupur (IN)

(21) Appl. No.: 19/303,812

(22) Filed: Aug. 19, 2025

Related U.S. Application Data
(60) Provisional application No. 63/685,193, filed on Aug. 20, 2024.

(51) Int. Cl. A61M 5/168

(52) U.S. Cl. CPC A61M 5/16881

(57) ABSTRACT

Flow path control devices and a method for delivering a medical fluid by flow path control device are disclosed. A flow path control device includes an inner housing and an outer housing. The inner housing includes a knob that rotates the inner housing relative to the outer housing. Rotating the knob activates different flow paths by aligning the flow paths with flow channels on the outer housing. Flow indicators on the inner housing are visible through windows in the outer housing and show a user of the flow path control device which flow channels are open. Additionally, at least one of the flow channels is an access port for a needleless fluid connector. When the flow channels are open, medical fluid can be transported through the flow channels.

### RELATED APPLICATIONS

This application claims priority to U.S. Provisional Application No. 63/685,193, which was filed on Aug. 20, 2024, and is entitled “NEEDLE FREE CONNECTOR CONTROL VALVE,” the entire disclosure of which is hereby incorporated by reference.

### TECHNICAL FIELD

The present disclosure relates generally to medical fluid valves and, more particularly, to valves modified to control medical fluid and connect to needleless fluid connector systems.

### BACKGROUND

Stopcocks are a device by which medical fluids are provided to patients, typically via medical infusion (i.e., intravenously). By using stopcocks, it is possible for multiple fluid sources and/or destinations to be connected. In an exemplary embodiment, a stopcock promotes fluid transmission between one or more medical fluid supplies and/or a patient's catheter line.

Needle-free connectors, including neutral displacement needle-free connectors, also offer a solution for providing medical fluid to patients. In an exemplary embodiment, a needle-free connector assembly promotes fluid transmission between a medical fluid supply and a catheter line. The medical fluid supply and the catheter line are secured to respective luers. To transmit fluid, the luer connected to the medical supply overlaps a central post of the luer connected to the catheter line.

### SUMMARY

Aspects of the present disclosure provide a flow path control device with an inner housing and an outer housing situated around the inner housing. The inner housing includes an actuator or knob that can be used to rotate the inner housing relative to the outer housing. By rotating the knob, a user of the flow path control device can connect flow paths on the inner housing to flow channels on the outer housing in a variety of combinations. As a result, medical fluids can be transported through different flow channels. Flow indicators on the inner housing show the user which flow channels are activated (i.e., which flow channels the medical fluid can is flowing in and out of). The user can view the flow indicator that correlates to the activated flow paths and/or flow channels through a window that is situated on the outer housing.

The present disclosure provides a multi-directional control valve that utilizes fewer parts (i.e., the inner housing and the outer housing) than stopcocks and integrates a needle-free fluid connector. Both factors reduce the number of potential failure points and the footprint of the device when compared to a stopcock, which, in turn, reduces errors associated with the use of stopcocks. For example, this design prevents infections (e.g., bloodstream infections), major blood loss, major drug loss and/or medication errors, and delays in delivery of medical fluids. The use of fewer parts and integration with a needleless fluid connector also reduces the bulk of the device. Reducing the bulk of the device makes it better suited for near-patient use, which is particularly useful where the fluid control device should be in close proximity to the patient (e.g., in intensive care units where several medical fluid lines need to be turned on and/or shut off quickly).

Aspects of the present disclosure provide for a flow path control device that includes a flow indicator. A flow indicator tells a user of the device which flow paths and flow channels are open for the reception and delivery of medical fluid. A flow indicator improves the user's intuitive understanding of the state of the valve (e.g., the flow path control device). This improved understanding of the state of the valve (i.e., which direction the medical fluid will flow in) reduces the likelihood of a user accidentally opening or closing a port, unknowingly failing to deliver a fluid, spilling fluid when a port is not attached to a fluid connection device, and/or improperly delivering fluid to a patient or other source.

Accordingly, aspects of the present disclosure provide a flow path control device for a fluid connector system, the device comprising: an outer housing having a plurality of flow channels configured to conduct fluid to or from the fluid flow path control device, at least one of the flow channels comprises an access port for a needleless fluid connector, and an inner housing, received within the outer housing, the inner housing comprising: a knob configured to rotate the inner housing relative to the outer housing; a plurality of flow paths, wherein rotating the inner housing aligns the plurality of flow paths to fluidly connect combinations of flow channels; flow indicators configured to indicate which combination of flow channels are fluidly connected, and wherein the outer housing comprises a window configured to display the flow indicator corresponding to the flow channels that are fluidly connected.

Some instances of the present disclosure provide a method for delivering a medical fluid, the method comprising, by a flow path control device: providing an inner housing, the inner housing comprising a knob, flow paths, and flow indicators; providing an outer housing, the outer housing comprising flow channels and a window; receiving, at one or more of the flow channels, a medical fluid; rotating, by the knob, the inner housing wherein rotating the knob comprises: displaying the desired flow indicator in the window, and aligning the flow paths with the desired flow channels; and transporting the medical fluid through the desired flow channels.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a perspective view of a first embodiment of a flow path control device, in accordance with some aspects of the present disclosure.

FIG. 2 illustrates a side view of a flow path control device, in accordance with some aspects of the present disclosure.

FIGS. 3A-3D illustrate top views of a flow path control device with different flow indicators displayed, in accordance with some aspects of the present disclosure.

FIGS. 3E-3H illustrate cross-sectional views of a flow path control device with different flow channels fluidly connected, in accordance with some aspects of the present disclosure.

FIG. 4 illustrates a perspective view of an inner housing of a flow path control device, in accordance with some aspects of the present disclosure.

FIG. 5 illustrates a perspective view of an outer housing of a flow path control device, in accordance with some aspects of the present disclosure.

FIG. 6 illustrates a perspective view of a second embodiment of a flow path control device, in accordance with some aspects of the present disclosure.

FIG. 7 illustrates a back view of a second embodiment of a flow path control device, in accordance with some aspects of the present disclosure.

FIG. 8 illustrates a perspective view of a third embodiment of a flow path control device, in accordance with some aspects of the present disclosure.

FIG. 9 illustrates a perspective view of a fourth embodiment of a flow path control device, in accordance with some aspects of the present disclosure.

FIG. 10 illustrates a flowchart showing a method for delivering a medical fluid, in accordance with some aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Needle-free connectors are essential devices to deliver fluid to a patient via an IV catheter. Needle-free connectors may be used in general patient populations, including neonatal, pediatric, and adult patients. In various applications, the pressure applied to the blood component should not exceed 300 mm Hg (5 psi) as this may result in hemolysis or bag breakage, the IV fluid needs to be injected in bolus without control during power injection and infusion pressure should never exceed 25 psi, as pressure higher than 25 psi may damage blood vessels. Thus, medical professional using needle-free connectors face challenges to maintain the various higher-pressure limits during infusion delivery with typical connectors.

Integrating needle-free connectors with typical stopcocks and/or other control valves has drawbacks. For example, an ordinary control valve would be too bulky when combined with a needleless fluid connector, which is not compatible with control valves that need to be near the patient (e.g., when a medication administered to the patient needs to be turned on or off quickly). Further, the bulk of typical stopcocks increase the amount of potential pressure points that may cause pain to the patient. The following devices and methods provide design modifications to overcome the foregoing issues.

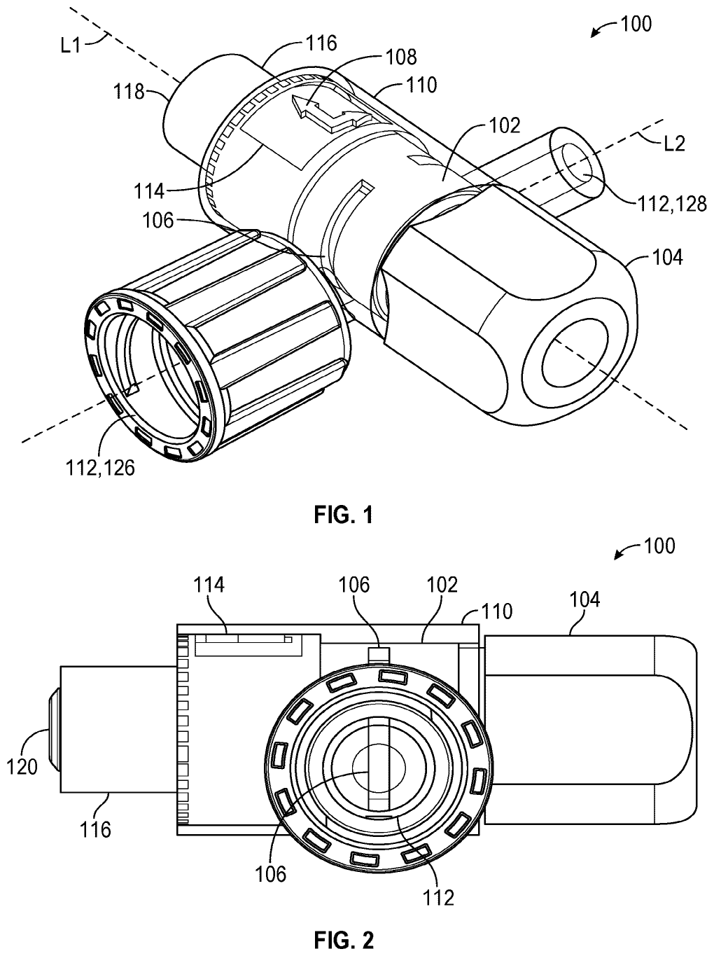

Referring now to the figures, FIG. 1 illustrates a perspective view of a first embodiment of a flow path control device 100, in accordance with some aspects of the present disclosure. The flow path control device 100 has an inner housing 102 and an outer housing 110. The inner housing 102 can include a knob 104, flow paths 106, and flow indicators 108. In some embodiments, the knob 104 is a handle, grip, crank, or shaft. A user of the flow path control device 100 can rotate, pivot, and/or twist the knob 104, which rotates, pivots, and/or twists the inner housing 102, and thus the flow paths 106 and the flow indicators 108, relative to the outer housing 110. In other words, the user can rotate the knob 104 to select the direction of the flow of medical fluid through the flow path control device 100. The outer housing 110 can include flow channels 112 and a window 114.

A flow path 106 is a route, passage, or track by which medical fluid flows, travels, or is transported between flow channels 112. When the user rotates the flow paths 106 via the knob 104, the user is aligning different flow paths 106 with different combinations of flow channels 112 to fluidly connect the different combinations of flow channels 112. When one or more flow channels 112 are fluidly connected or open, the medical fluid flows down the corresponding flow paths 106 and are transported between the desired and/or designated flow channels 112. The flow channels 112 direct the medical fluid to and/or from different sources. The flow channels 112 can act as access ports for other fluid connection devices. In some embodiments, the flow channels 112 are coupled to a patient by a catheter or other fluid connection device. In some embodiments, the flow channels 112 are coupled to an IV set by a catheter or other fluid connection device. In some embodiments, the flow channels 112 serve as a needleless fluid connector access port and are coupled to a needleless fluid connector assembly or system.

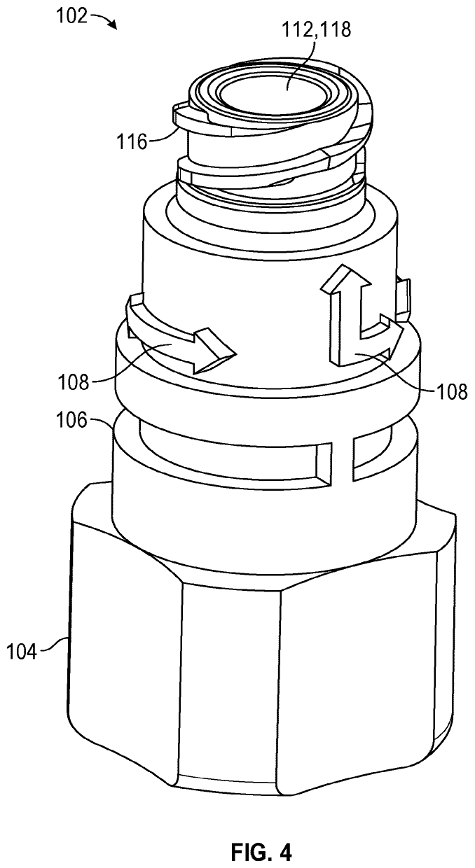

The inner housing 102 can include a center post mount 116. The center post mount 116 can include an inner volume 118 (shown in FIG. 4) that is occupied by a bellow 120 (shown in FIG. 2). The center post mount 116 can also function as a flow channel 112. The bellow 120 forms a seal at the entrance of the center post mount 116. A fluid connection device can push down on the bellow 120, which enables medical fluid to enter or exit the inner volume 118 of the center post mount 116. When the bellow 120 is compressed or pushed down, the inner volume 118 can act as one of the flow channels 112.

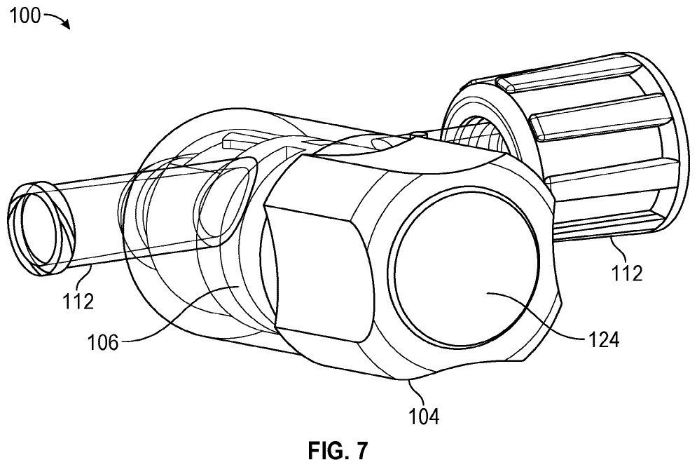

In some embodiments, the knob 104 is hollow and can also function as a flow channel 112. In some embodiments, the knob 104 is sealed with a plug 124 (shown in FIG. 7). The plug 124 can act as a seal and/or a stopper.

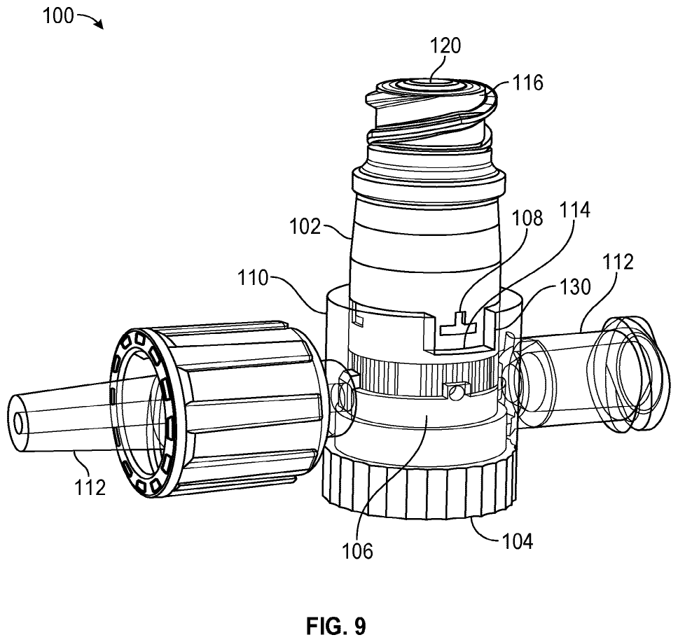

The flow indicators 108 show the user which combinations of flow channels 112 are fluidly connected. In some embodiments, the flow indicators 108 are arrows that point in the direction of the open flow channels 112. Other designs for the flow indicators 108 that indicate which flow channels 112 are open are also possible. For example, lines can be used in place of arrows (as shown in FIG. 9).

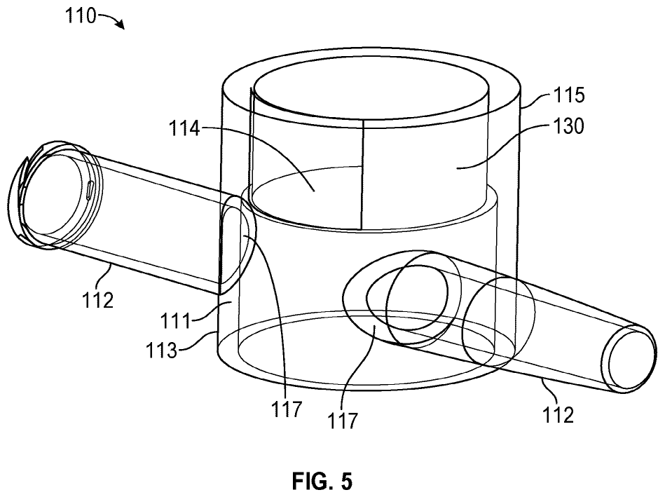

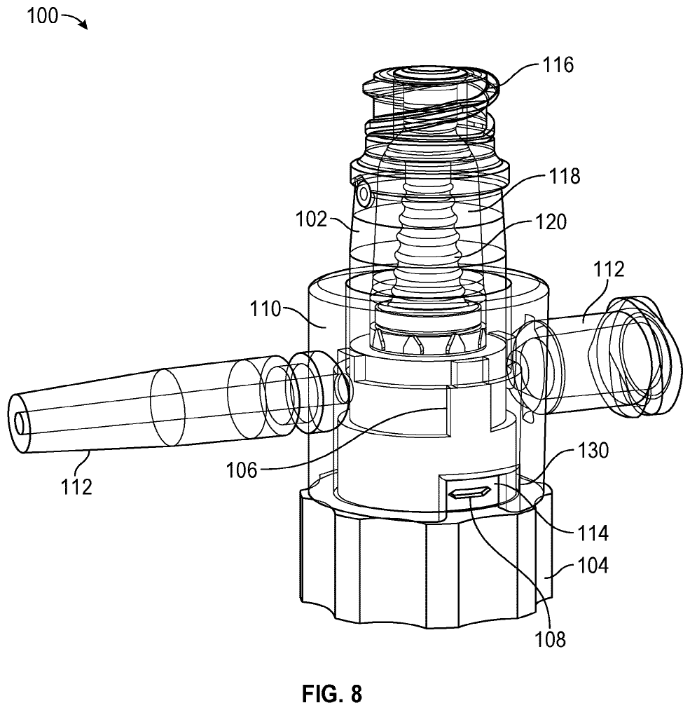

The flow indicators 108 are displayed in a window 114. In some embodiments, the window 114 is made of a transparent material and the region 130 surrounding the window (shown in FIG. 5) is made of an opaque material. In some embodiments, the window 114 is cut out of the region 130 that surrounds the window, and the region 130 can be made of a transparent or an opaque material (shown in FIGS. 8 and 9).

A first longitudinal axis L1 extends through the center of the center post mount 116 and the knob 104. A second longitudinal axis L2 extends through the center of the flow channels 112 on the side of the flow path control device 100. In FIG. 1, the second longitudinal axis L2 extends through the center of a first flow channel 126 and a second flow channel 128. The flow channels 112 are offset relative to the inner housing 102 (as further shown in FIG. 2), such that the first longitudinal axis L1 does not intersect with the second longitudinal axis L2.

FIG. 2 illustrates a side view of a flow path control device 100, in accordance with some aspects of the present disclosure. The side view of the flow path control device 100 shows how the flow path 106 aligns with the flow channel 112. When the flow channel 112 is open, the flow path 106 is lined up with the flow channel 112 such that the medical fluid can flow into and/or out of one flow channel 112, flow through a flow path 106, and flow out of and/or into another flow channel 112. The side view of the flow path control device 100 further demonstrates that some of the flow channels 112 are offset (i.e., not centered) relative to the inner housing. This side view also demonstrates that the window 114 only shows and/or displays one flow indicator 108 at a time (in particular, the flow indicator 108 that corresponds to the flow paths 106 that are activated and/or the flow channels 112 that are open and/or activated).

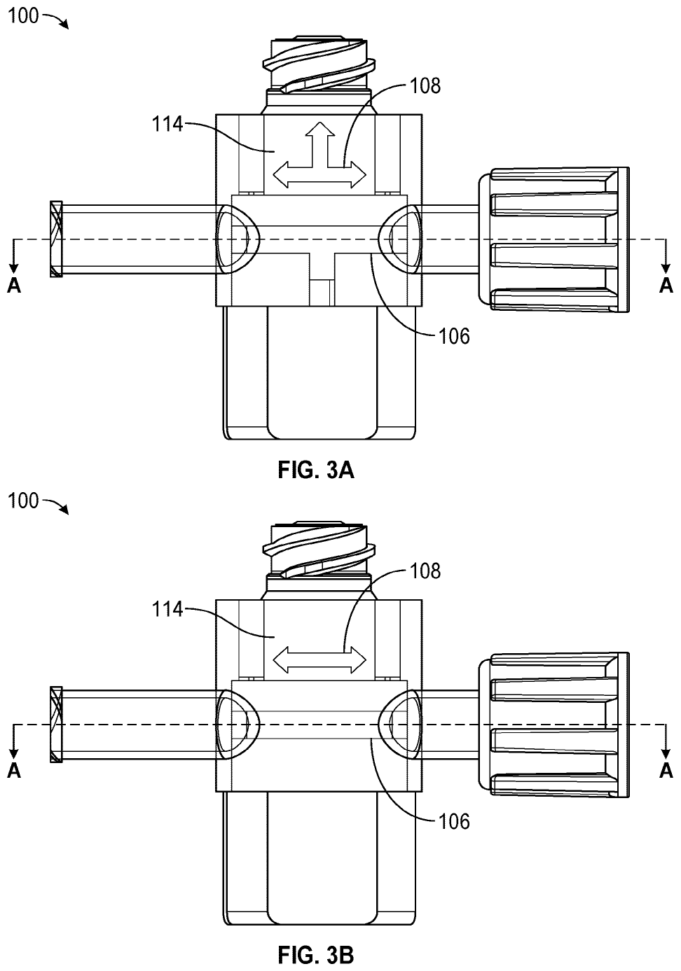

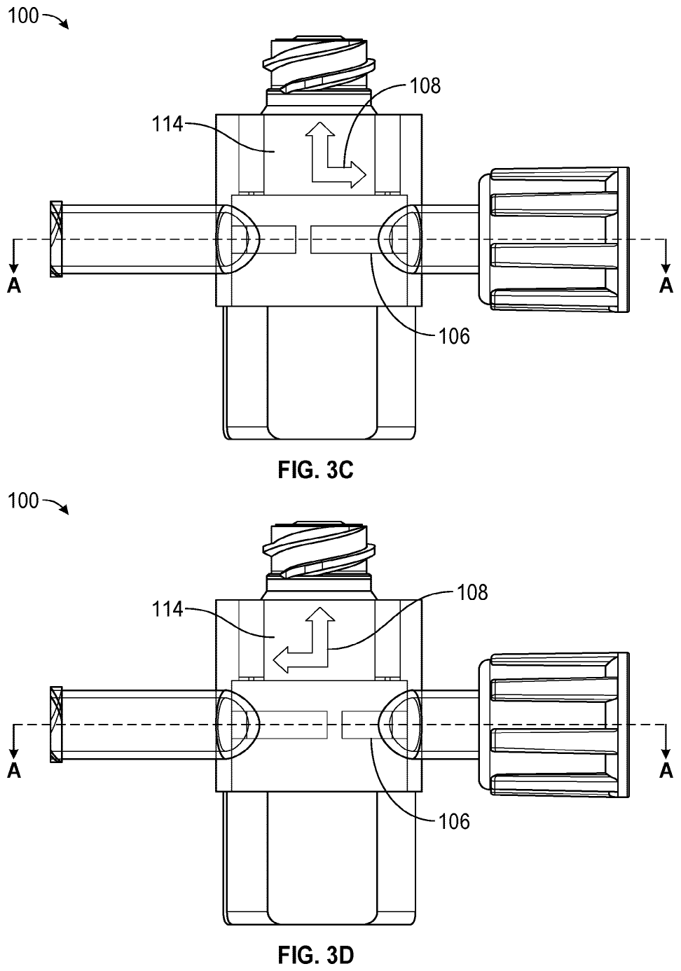

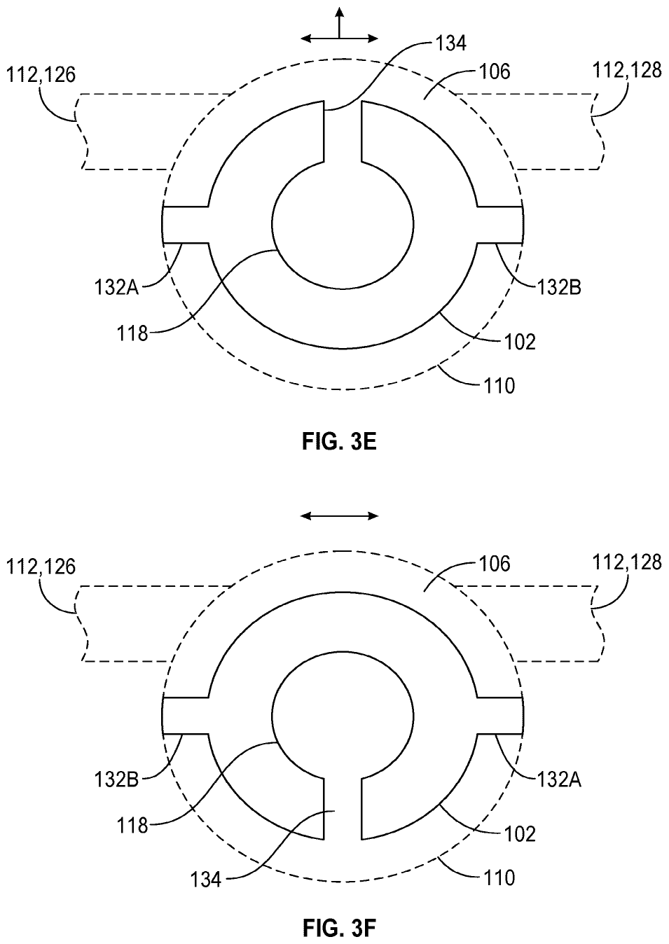

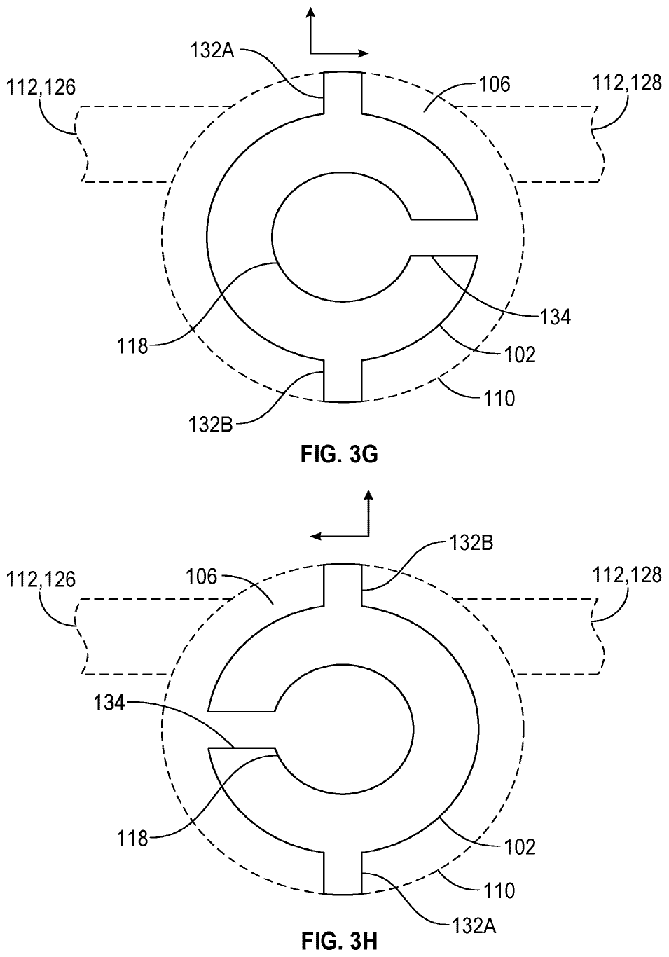

FIGS. 3A-3D illustrate top views of a flow path control device 100 with different flow indicators 108 displayed, in accordance with some aspects of the present disclosure. As previously described, the flow indicators 108 show a user of the flow path control device 100 which flow paths 106 are engaged and, thus, which flow channels 112 are open. In some embodiments, the flow path control device 100 includes four different combinations of flow channels 112 that can be fluidly connected at once: left, up, and right; left and right; up and right; and left and up. Other quantities and combinations of flow paths 106 are also possible.

FIGS. 3E-3H illustrate cross-sectional views of a flow path control device 100 with different flow channels 112 fluidly connected, in accordance with some aspects of the present disclosure. The cross section is taken from the dashed line A-A shown in FIGS. 3A-3D. The cross section shows that rotating the flow path 106 aligns different combinations of flow channels 112 by rotating the barriers 132A, 132B of the inner housing 102 and the aperture 134 of the inner housing to block and/or allow fluid flow between different combinations of flow channels 112.

Different flow paths 106 fluidly connect different combinations of flow channels 112. For example, in FIG. 3A, the flow indicator 108 shows that the left, top, and right flow channels 112 are open (or fluidly connected). As shown in FIG. 3E, the left, top, and right flow channels 112 are open because the inner housing 102 has been rotated such that the barriers 132A and 132B connect left flow channel 112 (also known as the first flow channel 126) and the right flow channel 112 (also known as the second flow channel 128). Simultaneously, the first flow channel 126 and the second flow channel 128 are fluidly connected to the top flow channel 112 (also known as the inner volume 118) via the aperture 134.

In FIG. 3B, the flow indicator 108 shows that the left and right flow channels 112 are open. As shown in FIG. 3F, the left and right flow channels 112 are open because the inner housing 102 has been rotated such that the barriers 132A and 132B connect the first flow channel 126 and the second flow channel 128. Meanwhile, the aperture 134 is not fluidly connected to the flow path 106, so the inner volume 118 is not fluidly connected to the first and second flow channels 126, 128.

In FIG. 3C, the flow indicator 108 shows that the left and top flow channels 112 are open. As shown in FIG. 3G, the barriers 132A and 132B obstruct flow between the first flow channel 126 and the second flow channel 128. At the same time, the aperture 134 is oriented such that the second flow channel 128 is fluidly connected to the inner volume 118.

Finally, in FIG. 3D, the flow indicator 108 shows that the right and top flow channels 112 are open. As shown in FIG. 3H, the barriers 132A and 132B obstruct flow between the first flow channel 126 and the second flow channel 128. At the same time, the aperture 134 is oriented such that the first flow channel 126 is fluidly connected to the inner volume 118.

FIG. 4 illustrates a perspective view of an inner housing 102 of a flow path control device 100, in accordance with some aspects of the present disclosure. The inner housing 102 has a cylindrical or generally cylindrical shape. Other shapes are also possible. The inner housing 102 can be tiered. The diameter of the inner housing 102 can change from the top of the inner housing 102 to the bottom of the inner housing 102. The inner housing 102 comprises the knob 104, the flow paths 106, the flow indicators 108, the center post mount 116, and the inner volume 118 of the center post mount 116.

The knob 104 has a cylindrical or generally cylindrical shape. Other shapes are also possible. In some embodiments, the knob 104 has ridges or grips.

In some embodiments, the flow paths 106 are notched, indented, and/or debossed in the inner housing 102. In some embodiments, the flow paths 106 are raised and/or embossed in the inner housing 102. Likewise, in some embodiments, the flow indicators 108 are notched, indented, and/or debossed in the inner housing 102. In some embodiments, the flow indicators 108 are raised and/or embossed in the inner housing 102. The flow indicators 108 can be arrows that point in the direction of the open flow channels 112. In some embodiments, the flow indicators 108 are lines that represent the direction that medical fluid can flow through the flow path control device 100. Other shapes of flow indicator 108 are also possible.

The center post mount 116 has a cylindrical or generally cylindrical shape. Other shapes are also possible. For example, the center post mount 116 can also have a tapered or conical shape. The center post mount 116 may comprise a threaded exterior. In some embodiments, the threaded exterior of the center post mount 116 is used to couple the flow path control device 100 to a fluid connector system. In some embodiments, the exterior of the center post mount 116 is smooth. The center post mount 116 may have an inner volume 118, which can serve as a flow channel 112. In some embodiments, a bellow 120 fills the inner volume 118 (as best shown in FIG. 8). Where the inner housing 102 includes a bellow 120, the bellow 120 functions as a seal for the center post mount 116, preventing medical fluid from entering the inner volume 118. When the bellow 120 is depressed, compressed, and/or pushed, the inner volume 118 is exposed, and medical fluid can enter the inner volume 118.

In some embodiments, the center post mount 116 is a boxer female luer. The user can connect a male luer to the flow path control device 100 at the boxer female luer.

FIG. 5 illustrates a perspective view of an outer housing 110 of a flow path control device 100, in accordance with some aspects of the present disclosure. The outer housing 110 has a central body 115 and flow channels 112, which protrude from the central body 115. The central body 115 has a cylindrical or generally cylindrical shape. Other shapes are also possible. The central body 115 comprises a hollow interior. When the flow path control device 100 is assembled, the inner housing 102 occupies the hollow interior of the central body 115 of the outer housing 110.

The wall of the central body 115 can comprise any thickness or thinness. The wall of the central body 115 comprises an interior surface 111 and an exterior surface 113. In some embodiments, the wall of the central body 115 is a double-paned wall that is hollow between the two walls. Where the wall of the central body 115 is a double-paned wall, the interior surface 111 is an interior wall and the exterior surface 113 is an exterior wall.

The central body 115 can be made from a transparent material and/or an opaque material. However, the portion of the central body 115 that comprises the window 114 must be made from a transparent material or be cut or carved out of the material that comprises the central body 115. In some embodiments, the region 130 that surrounds the window 114 is made from a different material than the remainder of the central body 115.

The flow channels 112 protrude from the central body 115 of the outer housing 110. In some embodiments, the flow channels 112 have a cylindrical or generally cylindrical shape. In some embodiments, the flow channels 112 have a conical or generally conical shape. In some embodiments, the flow channels 112 have different shapes from each other. Other shapes are also possible.

The central body 115 has one or more cutouts, bores, and/or holes 117. The flow channels 112 protrude from the holes in the central body 115. The holes 117 connect the flow paths 106 to the flow channels 112. When the user of the flow path control device 100 rotates the inner housing 102, different flow paths 106 and/or different parts of the flow paths 106 align with the holes 117 to open different combinations of flow channels 112. When the flow paths 106 are aligned with the holes 117, medical fluid can flow between the flow channels 112 via the flow paths 106.

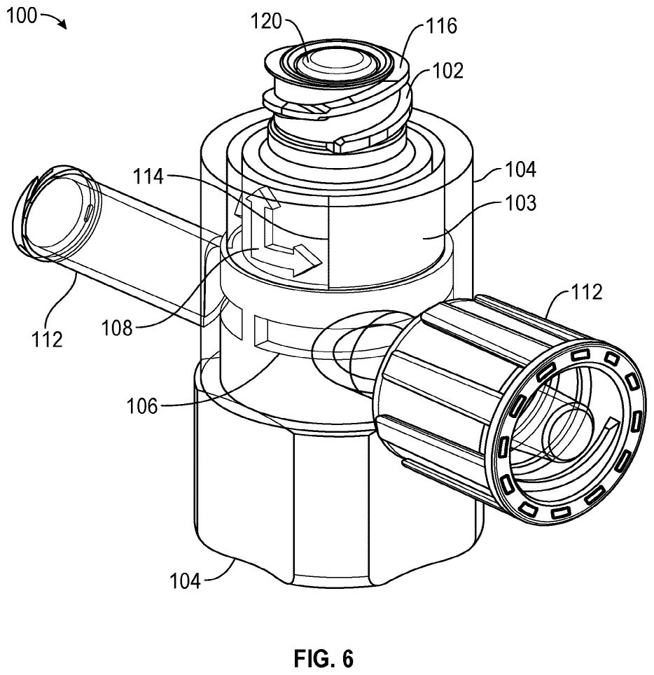

FIG. 6 illustrates a perspective view of a second embodiment of a flow path control device 100, in accordance with some aspects of the present disclosure. In this embodiment, the center post mount 116 is has a threaded exterior instead of a smooth exterior. Additionally, in this embodiment, the inner housing 102 (in particular, the center post mount 116, the flow indicators 108, the flow paths 106, and the knob 104) comprises a singular, unified part.

FIG. 7 illustrates a back view of a second embodiment of a flow path control device 100, in accordance with some aspects of the present disclosure. In this embodiment, the knob 104 has a hollow interior that is occupied by a sealing plug 124.

FIG. 8 illustrates a perspective view of a third embodiment of a flow path control device 100, in accordance with some aspects of the present disclosure. In this embodiment, the center post mount 116 is made of a transparent material, and the bellow 120 that occupies the inner volume 118 is visible from outside the flow path control device 100. Also in this embodiment, the window 114 is situated immediately above the knob 104. The window 114 is cut out of the outer housing 110 (whereas in previous embodiments, the window 114 comprised a transparent material). This embodiment further features flow indicators 108 integrated as part of the knob 104, with the flow indicators 108 beneath the flow paths 106. Finally, this embodiment demonstrates a different design of the flow indicators 108 and a different configuration for the flow paths 106.

FIG. 9 illustrates a perspective view of a fourth embodiment of a flow path control device 100, in accordance with some aspects of the present disclosure. This embodiment demonstrates the window 114 placed at the top of the outer housing 110 and cut out of the outer housing 110. This embodiment further demonstrates a different design of the flow indicators 108 and a different configuration for the flow paths 106.

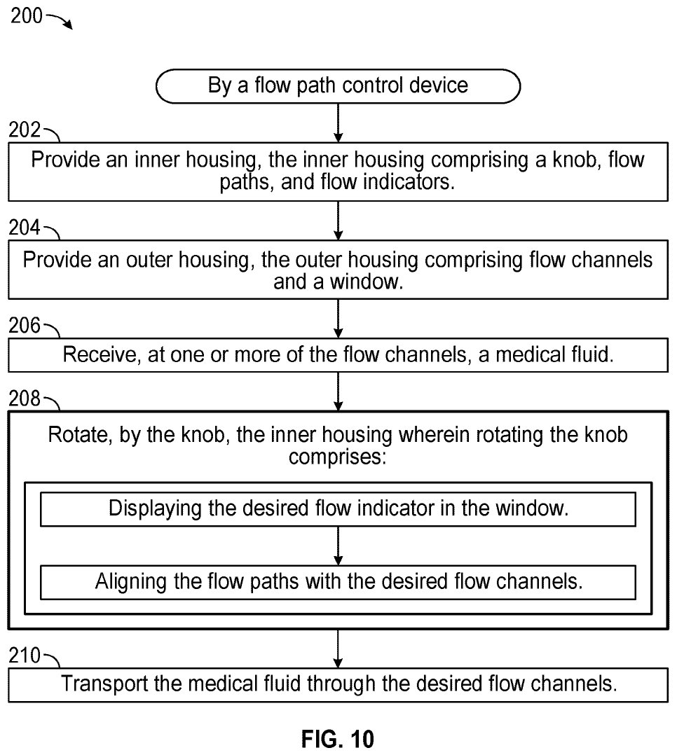

FIG. 10 illustrates a flowchart 200 showing a method for delivering a medical fluid, in accordance with some aspects of the present disclosure. The method shown in the flowchart 200 may be performed by flow path control devices described herein. Accordingly, flow path control devices described herein can carry out the method shown in the flowchart 200.

In step 202, an inner housing is provided. The inner housing comprises a knob, flow paths, and flow indicators. The knob can be used to rotate the inner housing. Rotating the inner housing engages different flow paths and displays different flow indicators. The flow indicators show a user of the flow path control device which of the flow paths are engaged.

In step 204, an outer housing is provided. The outer housing comprises flow channels and a window. The window frames and/or displays the flow indicators.

In step 206, a medical fluid is received at one or more of the flow channels. In some embodiments, the medical fluid is received by way of a needleless fluid connector.

In step 208, the inner housing, by way of the knob, is rotated. Rotating the inner housing changes the flow indicator that is displayed in the window. The flow indicators show a user of a flow path control device which flow paths are engaged and/or which flow channels are open. Rotating the inner housing engages different combinations of flow paths, which opens different combinations of flow channels.

In step 210, the medical fluid is transported through the desired flow channels. Depending on which flow paths are engaged and/or which flow channels are opened, the flow path control device will transport the medical fluid to and from different flow channels.

Although the present disclosure includes embodiments in which a flow path control device includes a three flow channels in the Figures, it should be understood that the post may include any number of flow channels, each of which can receive and/or transport a fluid from a fluid delivery device.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. A flow path control device for a fluid connector system, the device comprising: an outer housing having a plurality of flow channels configured to conduct fluid to or from the fluid flow path control device, at least one of the flow channels comprises an access port for a needleless fluid connector, and an inner housing, received within the outer housing, the inner housing comprising: a knob configured to rotate the inner housing relative to the outer housing; a plurality of flow paths, wherein rotating the inner housing aligns the plurality of flow paths to fluidly connect combinations of flow channels; flow indicators configured to indicate which combination of flow channels are fluidly connected, and wherein the outer housing comprises a window configured to display the flow indicator corresponding to the flow channels that are fluidly connected.

Clause 2. The device of clause 1, wherein the inner housing comprises: a center post mount with an inner volume; a bellow located in the inner volume of the center post mount, wherein the center post mount comprises one of the flow channels when the bellow is compressed.

Clause 3. The device of clause 1, wherein a bottom of the inner housing is sealed with a plug.

Clause 4. The device of clause 1, wherein at least a first longitudinal axis of a first flow channel does not intersect with a second longitudinal axis of a second flow channel.

Clause 5. The device of clause 1, wherein the window comprises a transparent material.

Clause 6. The device of clause 1, wherein the window comprises a portion that is cut out of the outer housing.

Clause 7. The device of clause 1, wherein a region of the outer housing that surrounds the window comprises an opaque material.

Clause 8. The device of clause 1, wherein a region of the outer housing that surrounds the window comprises a transparent material.

Clause 9. A method for delivering a medical fluid, the method comprising, by a flow path control device: providing an inner housing, the inner housing comprising a knob, flow paths, and flow indicators; providing an outer housing, the outer housing comprising flow channels and a window; receiving, at one or more of the flow channels, a medical fluid; rotating, by the knob, the inner housing wherein rotating the knob comprises: displaying the desired flow indicator in the window, and aligning the flow paths with the desired flow channels; and transporting the medical fluid through the desired flow channels.

Clause 10. The method of clause 9, wherein receiving a medical fluid comprises connecting a needleless fluid connector with one or more of the flow channels.

Clause 11. The method of clause 9, wherein receiving a medical fluid comprises:
providing, in one or more of the flow channels, a bellow, forming, with the bellow, a seal at a proximal end of the one or more flow channels, and compressing the bellow towards a distal end of the one or more flow channels to allow fluid to enter the one or more flow channels.

Clause 12. The method of clause 9, wherein displaying the desired flow indicator in the window comprises improving an understanding of the state of the flow path control device for a user of the flow path control device.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more. ” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration. ” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled”or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims.

Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A flow path control device, the device comprising:
an outer housing having a plurality of flow channels configured to conduct fluid to or from the fluid flow path control device, at least one of the flow channels comprises an access port for a needleless fluid connector, and an inner housing, received within the outer housing, the inner housing comprising:
a knob configured to rotate the inner housing relative to the outer housing;
a plurality of flow paths, wherein rotating the inner housing aligns the plurality of flow paths to fluidly connect combinations of flow channels;
flow indicators configured to indicate which combination of flow channels are fluidly connected, and
wherein the outer housing comprises a window configured to display the flow indicator corresponding to the flow channels that are fluidly connected.

2. The device of claim 1, wherein the inner housing comprises:
a center post mount with an inner volume;
a bellow located in the inner volume of the center post mount, wherein the center post mount comprises one of the flow channels when the bellow is compressed.

3. The device of claim 1, wherein a bottom of the inner housing is sealed with a plug.

4. The device of claim 1, wherein at least a first longitudinal axis of a first flow channel does not intersect with a second longitudinal axis of a second flow channel.

5. The device of claim 1, wherein the window comprises a transparent material.

6. The device of claim 1, wherein the window comprises a portion that is cut out of the outer housing.

7. The device of claim 1, wherein a region of the outer housing that surrounds the window comprises an opaque material.

8. The device of claim 1, wherein a region of the outer housing that surrounds the window comprises a transparent material.

9. A method for delivering a medical fluid, the method comprising, by a flow path control device:
providing an inner housing, the inner housing comprising a knob, flow paths, and flow indicators;
providing an outer housing, the outer housing comprising flow channels and a window;
receiving, at one or more of the flow channels, a medical fluid;
rotating, by the knob, the inner housing wherein rotating the knob comprises:
displaying the desired flow indicator in the window, and
aligning the flow paths with the desired flow channels; and
transporting the medical fluid through the desired flow channels.

10. The method of claim 9, wherein receiving a medical fluid comprises connecting a needleless fluid connector with one or more of the flow channels.

11. The method of claim 9, wherein receiving a medical fluid comprises:
providing, in one or more of the flow channels, a bellow,
forming, with the bellow, a seal at a proximal end of the one or more flow channels, and
compressing the bellow towards a distal end of the one or more flow channels to allow fluid to enter the one or more flow channels.

12. The method of claim 9, wherein displaying the desired flow indicator in the window comprises improving an understanding of the state of the flow path control device for a user of the flow path control device.
